(19) United States
(12) Patent Application Publication
(10) Pub. No.: US 2024/0218389 A1
(43) Pub. Date: Jul. 4, 2024

(54) METHODS FOR CONSTRUCTING TRANSGENIC PLANTS COMPRISING TaPDIL4-1B GENE

(71) Applicant: INSTITUTE OF GRAIN CROPS, HUBEI ACADEMY OF AGRICULTURAL SCIENCES, Wuhan (CN)

(72) Inventors: Yike LIU, Wuhan (CN); Zhanwang ZHU, Wuhan (CN); Bo WEI, Wuhan (CN); Jinghan SONG, Wuhan (CN); Juan ZOU, Wuhan (CN); Hanwen TONG, Wuhan (CN); Ling CHEN, Wuhan (CN); Chunbao GAO, Wuhan (CN)

(73) Assignee: INSTITUTE OF GRAIN CROPS, HUBEI ACADEMY OF AGRICULTURAL SCIENCES, Wuhan (CN)

(21) Appl. No.: 18/611,679

(22) Filed: Mar. 20, 2024

Related U.S. Application Data
(63) Continuation of application No. PCT/CN2023/101955, filed on Jun. 21, 2023.

(30) Foreign Application Priority Data
Jun. 28, 2022 (CN) 202210747589.6

(51) Int. Cl. C12N 15/82; A01H 6/46

(52) U.S. Cl. CPC C12N 15/8282; A01H 6/4678

(57) ABSTRACT

The embodiments of the present disclosure relate to the field of genetic engineering technology, and in particular, to a method for constructing a transgenic plant comprising a TaPDIL4-1B gene. A cDNA sequence of the TaPDIL4-1B gene is shown in SEQ ID NO: 1. The embodiments of the present disclosure apply the genetic engineering technology to obtain the TaPDIL4-1B gene from wheat for the first time, and transfer the gene into plants through Agrobacterium-mediated manners. This gene can make plants have strong resistance to scab, which provides good candidate genes for the development and breeding of new crop varieties with resistance to fusarium head blight.

### CROSS-REFERENCE TO RELATED APPLICATIONS

This application is a continuation of International Patent Application No. PCT/CN2023/101955, filed on Jun. 21, 2023, which claims priority to Chinese Patent Application No. 202210747589.6, filed on Jun. 28, 2022, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Mar. 5, 2024, is named “2024-03-20-Sequence listing-67601-H001US00” and is 14,815 bytes in size.

### TECHNICAL FIELD

The present disclosure relates to the field of genetic engineering technology, and in particular, to a method for constructing a transgenic plant comprising a TaPDIL4-1B gene.

### BACKGROUND

Wheat Fusarium Head Blight (FHB) is a global fungal disease caused by Fusarium species, which has been increasing in frequency and expanding in scope with global warming and changes in farming practices. FHB not only severely affects wheat yield, but Fusarium species also produce a large amount of mycotoxins during wheat infestation. If wheat (also referred to as Triticum aestivum) and its products contaminated with toxins are consumed by humans and animals, it can cause vomiting, diarrhea, miscarriage, and other issues, seriously endangering human and animal health. FHB has a significant impact on food production and food safety and has become a focus of international concern.

In genetic improvement of wheat FHB, there are limited primary genes that can be utilized, and the resistance mechanism of some genes is still unclear, leading to slow progress in improving resistance to wheat FHB and difficulty in meeting production demands. Therefore, cloning new genes capable of resisting wheat FHB and studying their molecular mechanisms is of great significance. Genetic improvement of wheat resistance to wheat FHB based on these new genes is important.

### SUMMARY

Embodiments of the present disclosure provide a method for constructing a transgenic plant comprising a TaPDIL4-1B gene, wherein a cDNA sequence of the TaPDIL4-1B gene may be shown in SEQ ID NO:1.

In some embodiments, the plant may be wheat.

In some embodiments, the TaPDIL4-1B gene or a recombinant plasmid comprising the TaPDIL4-1B gene may be introduced into cells, tissues, or organs of the wheat to obtain a new wheat variety with resistance to fusarium head blight.

In some embodiments, the recombinant plasmid may be pMWB110-TaPDIL4-1B.

In some embodiments, the recombinant plasmid pMWB110-TaPDIL4-1B may be selectively labeled.

### BRIEF DESCRIPTION OF THE DRAWINGS

FIG. 1 is a Polymerase Chain Reaction (PCR) detection result of a transgenic wheat positive plant comprising a TaPDIL4-1B gene according to some embodiments of the present disclosure, where #1-#8 are transgenic plants, Fielder is a wild-type plant, and NC is a negative control where an amplification template is water.

FIG. 2 is a comparison of FHB resistance of overexpression plants 21 days after inoculation and the wild-type plant Fielder according to some embodiments of the present disclosure, where (A) is a phenotypic identification result of FHB resistance of transgenic wheat comprising the TaPDIL4-1B gene (which was obtained by using single flower drip manner to inoculate wheat ears and recording the disease incidence); and (B) is a statistical result of an incidence rate of FHB in spikelets of the transgenic wheat comprising the TaPDIL4-1B gene. T is the transgenic plant and CK is the wild-type plant Fielder.

### DETAILED DESCRIPTION

As used in the present disclosure and the claims, the singular forms “a,” “an,” and/or “the” include plural referents unless the context clearly indicates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may further include other steps or elements.

Unless otherwise defined, all technical and scientific terms used herein have the same meanings as those commonly understood by those of ordinary skill in the art to which this present disclosure belongs.

The results of the current study suggest that reactive oxygen species homeostasis (ROS homeostasis) and programmed cell death are associated with wheat FHB resistance. Two reductase systems comprising thioredoxin (Trx) and glutaredoxin (Grx) can regulate each other and serve a variety of roles such as scavenging reactive oxygen species and maintaining redox balance in organisms. Protein disulfide isomerase (PDI) includes a variable number and variety of Trx structural domains and belongs to the thioredoxin superfamily, and PDI family genes in plants are generally named protein disulfide isomerase-like (PDIL), and current functional studies of this class of genes are mainly focused on the formation of wheat quality. As of this writing, there are no reports on the involvement of the wheat PDIL gene in the regulation of wheat FHB resistance.

Carrying out the discovery of genes for resistance to wheat FHB is one of the most effective defense and control ways to control FHB.

Embodiments of the present disclosure provide a method for constructing a transgenic plant comprising a TaPDIL4-1B gene, and a cDNA sequence of the TaPDIL4-1B gene is shown in SEQ ID NO:1.

In some embodiments, the plant is wheat.

In some embodiments, the method includes: introducing the TaPDIL4-1B gene or a recombinant plasmid comprising the TaPDIL4-1B gene into cells, tissues, or organs of the wheat to obtain a new wheat variety with resistance to fusarium head blight.

In some embodiments, the recombinant plasmid is pMWB110-TaPDIL4-1B.

In some embodiments, the method further includes: selectively labeling the recombinant plasmid pMWB110-TaPDIL4-1B.

Selective labeling is a labeling manner used in a breeding process, which helps to distinguish and screen out cells, tissues, or organs carrying a target gene or trait by introducing a specific gene or labeled gene into an expression vector. The use of selective labeling improves the screening efficiency during the breeding process and ensures that only plants possessing the target trait survive and grow, thereby promoting stable inheritance of disease resistance traits and the breeding of new varieties.

Selective labeling may include the following steps:
1. selecting a suitable labeled gene. The labeled gene is a gene with a known function or a known sequence that can act as a specific label. In genetic engineering, the labeled gene is an important label for recombinant DNA vectors, and are often used to test whether the target gene has been successfully transformed.

In some embodiments, the labeled gene includes a β-glucosidase (GUS) gene.
2. constructing the TaPDIL4-1B gene and the labeled gene into the recombinant plasmid pMWB110-TaPDIL4-1B and introducing the recombinant plasmid into the cells, tissues, or organs of the wheat by appropriate transformation manners (e.g., gene gun manner or Agrobacterium-mediated manner).
3. at a culture stage after introduction, identifying whether the gene TaPDIL4-1B is successfully transformed into the cells, tissues, or organs of the wheat by, for example, detecting GUS activity.

The present disclosure provides an application of the TaPDIL4-1B gene in plant resistance to FHB, the cDNA sequence of the TaPDIL4-1B gene is shown in SEQ ID NO: 1.

In some embodiments, a gDNA sequence of the TaPDIL4-1B gene is shown in SEQ ID NO: 2, and the gDNA is composed of 11 exons and 10 introns; starting from the 5′ end, lengths of the exons are 224 bp, 85 bp, 98 bp, 56 bp, 118 bp, 28 bp, 92 bp, 157 bp, 125 bp, 100 bp, and 488 bp, respectively, and lengths of the introns are 1131 bp, 143 bp, 85 bp, 422 bp, 96 bp, 115 bp, 216 bp, 86 bp, 122 bp, and 88 bp, respectively.

In some embodiments, an amino acid sequence of an encoded protein of the TaPDIL4-1B gene is shown in SEQ ID NO:3. The protein is a thiodisulfide bond oxidoreductase located in the endoplasmic reticulum.

Embodiments of the present disclosure provide a recombinant plasmid, the recombinant plasmid comprising an FHB-resistant gene, i.e., TaPDIL4-1B; the vector of the plasmid is preferably pMWB110, i.e., the recombinant plasmid is preferably pMWB110-TaPDIL4-1B. Furthermore, the present disclosure does not limit the selection of vectors, any kind of vector that can introduce an exogenous gene into a plant for expression may be used in the present disclosure.

Embodiments of the present disclosure provide an application of the TaPDIL4-1B gene in the selection of plant varieties for resistance to FHB, and the cDNA sequence of the TaPDIL4-1B gene is shown in SEQ ID NO:1.

The beneficial effects of the embodiments of the present disclosure include, but are not limited to: (1) applying plant genetic engineering technology, the TaPDIL4-1B gene was obtained for the first time from a variety of wheat named “Chinese Spring”, and through experiments, it has been proven that the gene can be transferred into plants (wheat) through the Agrobacterium-mediated manner, which can give the plants (wheat) strong resistance to FHB, which provides a good candidate gene for researching and breeding new crop varieties resistant to FHB; and (2) the new FHB-resistant gene TaPDIL4-1B is of great significance for the genetic improvement of wheat FHB resistance, and is suitable for popularization and application.

The technical solutions in the embodiments of the present disclosure will be clearly and completely described below in conjunction with the accompanying drawings in the embodiments of the present disclosure, and it is clear that the embodiments described are only a portion of the embodiments of the present disclosure and not all of the embodiments. Based on the embodiments in the present disclosure, all other embodiments obtained by a person of ordinary skill in the art without making creative labor fall within the scope of protection of the present disclosure.

The experimental methods used in the following embodiments are conventional if not otherwise specified. The experimental materials, reagents, and the like used in the embodiments are commercially available if not otherwise specified. The quantitative tests in the following embodiments are set up for three repetitions of the experiment, and the results are averaged. M in the present disclosure denotes mol/L.

The wheat variety used to obtain the TaPDIL4-1B gene in the present disclosure is “Chinese Spring”, which is a very important local variety of wheat. The “Chinese Spring” wheat is widely used in wheat genetics research. The wheat variety used in the genetic modification or transgenic experiments of the embodiments of the present disclosure is a variety named Fielder.

### EMBODIMENTS

### Embodiment 1: Cloning of the TaPDIL4-1B Gene

### 1. Extraction of Total Wheat RNA

(1) Placing a tissue material of the wheat variety “Chinese Spring” in a mortar pre-cooled with liquid nitrogen and grinding to a powder;
(2) when the liquid nitrogen evaporates and dries up, immediately transferring the powder to a centrifuge tube of 2 mL, adding about 1 mL of Invitrogen's Trizol extract into every 100 mg of material as a sample, and after melting, repeatedly aspirating and blowing the sample with a spiking gun, mixing the sample with vigorous shaking to allow for full cleavage, and leaving the sample at room temperature for 5 minutes;
(3) adding chloroform of 0.2 mL, mixing with vigorous shaking for 15 seconds, and leaving it at room temperature for 10 minutes; and centrifuging with 12,000 rpm for 15 minutes at 4° C.;
(4) carefully aspirating an upper aqueous phase, adding the upper aqueous phase to a clean centrifuge tube of 1.5 mL, adding isopropanol of 500 μL (wherein a volume ratio of the upper aqueous phase to the isopropanol is 1:1), mixing thoroughly, and precipitating for 30 minutes at −20° C.; and centrifuging for 10 minutes with 12,000 rpm at 4° C., carefully discarding supernatant, and keeping precipitate;
(5) washing the precipitate with an ethanol solution with a volume percent concentration of 75% of 1 mL, and collecting RNA precipitate by centrifuging with 8000 rpm for 10 minutes at 4° C.;
(6) drying the RNA precipitate on a sterile bench for about 10-15 minutes, when the RNA was slightly transparent, adding RNase-free water of 50 μL to fully dissolve it, which may be stored at −80° C. for long-term storage and use; and
(7) detecting an RNA concentration and quality by an ultraviolet spectrophotometer and Agarose gel with a mass percent concentration of 1% through electrophoresis.
   2. Reverse Transcription of cDNA

The reverse transcription was carried out according to the instructions of RNAPCRKit (AMV) Ver.3.0 kit (TaKaRa, DRR019A). A reverse transcription reaction was first performed, and RNA of 500 ng was used as a template for the reverse transcription. Before the reverse transcription reaction, MgCl₂, 10×RT buffer, RNase-free H₂O, dNTP Mixture, RNase Inhibitor, AMV Reverse Transcriptase, Oligo dT Primer, and Total RNA were added to a reaction system, and a total volume of the reaction system was 10 μL. The reaction program was as follows: 42° C. for 30 minutes, 99° C. for 5 minutes, and 5° C. for 5 minutes.

### 3. Cloning and Sequencing

(1) Using cDNA as a template, designing gene-specific primers in 5′ UTR and 3′ UTR with primer sequences (as shown in SEQ ID NO: 4-5), respectively, including:

upstream primer F1:
(SEQ ID NO: 4)
5′-GAGCTCGCAGCAAAACAGAT-3′;
and
downstream primer R1:
(SEQ ID NO: 5)
5′-CCGCTAAACTTTCACTGCCA-3′;

(2) a PCR reaction system (20 UL in total) composed of:
2×TaqPCR MasterMix, 10 μL;
upstream primer F1, 0.5 μL;
downstream primer R1, 0.5 μL;
template cDNA, 1 μL; and
ddH₂O, 8 μL;
(3) a PCR reaction program including: pre-denaturation at 94° C. for 5 minutes; denaturation at 94° C. for 30 sec, annealing at 58° C. for 30 sec, extension at 72° C. for 1 minute and 15 seconds, 30 cycles/min; extension at 72° C. for 7 minutes; storing at 20° C., and amplifying an obtained specific amplified band.

A recovered PCR product was connected to pMT18-T (Bao Biotechnology Co., Ltd.) for sequencing, and a size from a start codon to a stop codon of a nucleotide sequence of the PCR product obtained by amplification was 1104 bp (shown in SEQ ID NO: 1), and the gene of the PCR product was named TaPDIL4-1B, and an amino acid sequence of an encoded protein of the TaPDIL4-1B gene is shown in SEQ ID NO: 3.

(SEQ ID NO: 1)
ATGGCGACCCCTCAGATCTACCGCAAAACCCTCCTCCCCGCCTAC
TCCTGCTCGCGGCCGCGGCGCTCTACCCAGCCGCCGCCGACGGC
GACGAGGTGCTCGCCCTCACGGAGTCCACCTTCGAGAAGGAGGTC
GGCCAGGACCGTGGCGCGCTCGTCGAGTTCTACGCTCCCTGGTGT
GGCCACTGCAAGAAGCTTGCTCCGGAATATGAGAAGCTTGCTGCA
AGTTTTAAAAAGGCTAAATCAGTCTTGATTGCCAAGGTTGACTGC
GATGAGCACAAGAGTGTGTGCAGCAAGTATGGAGTTTCTGGCTAC
CCCACAATCCAGTGGTTTCCCAAAGGTTCCTTGGAGCCCAAGAAG
TATGAGGGTCAACGCACTGCTGAAGCCCTTACAGAATATGTTAAC
TCTGAAGCAGCAACCAATGTGAAGATAGCAGCAGTTCCTTCAAGT
GTTGTGGTTCTGACCGAGGAAACCTTTGACTCAGTTGTCCTTGAT
GAAACCAAAGATGTCCTTGTTGAGTTCTATGCCCCATGGTGTGGT
CACTGCAAGAGTCTTGCTCCGATATATGAGAAGGTGGCCTCTGTT
TTCAAGCAAGATGAGGGTGTTGTGATTGCTAATCTTGATGCTGAC
AAATACACAAGCTTGGCTGAGAAGTATGGAGTTTCTGGTTTTCCC
ACATTGAAGTTCTTCCCGAAGGGTAACAAAGCTGGTGAAGAGTAT
GAGAGCGGTCGGGAGTTAGATGACTTTGTTAAGTTCATTAATGAG
AAAAGTGGAACTAGCCGTGATTCGAAGGGTCAGCTTACTTCAGAG
GCTGGGCTTGTGGCAAGTTTGGATGCTCTTGTCAAGGAATTTCAC
AGTGCTGCTGATGACAAGCGGAAGGAAATCCTCTCTAAAATTGAA
GAGGAGGCTGCGAAGCTCAGTGGTCCTGCTGTCAAGCACGGAAAG
ATCTATGTGAATGTTGCAAAGAAGATATTGCAGAAGGGCTCTGAC
TATACCAAGAAGGAAACTGAGAGGCTTCATCGCTTGTTGGAGAAG
TCGATCAGTCCTTCCAAAGCCGATGAATTCGCCATCAAGAAGAAC
ATTCTTTCAGCCTTCTCCTCTTAA;
(SEQ ID NO: 3)
MATPQIYRKTLLPVLLLLAAAALYPAAADGDEVLALTESTFEKEV
GQDRGALVEFYAPWCGHCKKLAPEYEKLAASFKKAKSVLIAKVD
CDEHKSVCSKYGVSGYPTIQWFPKGSLEPKKYEGQRTAEALTEYV
NSEAATNVKIAAVPSSVVVLTEETFDSVVLDETKDVLVEFYAPWC
GHCKSLAPIYEKVASVFKQDEGVVIANLDADKYTSLAEKYGVSGF
PTLKFFPKGNKAGEEYESGRELDDFVKFINEKSGTSRDSKGQLTS
EAGLVASLDALVKEFHSAADDKRKEILSKIEEEAAKLSGPAVKHG
KIYVNVAKKILQKGSDYTKKETERLHRLLEKSISPSKADEFAIKK
NILSAFSS.

### Embodiment 2: Cloning of Full-Length TaPDIL4-1B Genomic DNA (i.e., Full-Length gDNA)

(1) gDNA extraction: extracting gDNA of “Chinese Spring” wheat according to Plant gDNA extraction kit instructions (Tiangen Biochemical Technology Co., Ltd., Beijing);
(2) using the gDNA of “Chinese Spring” wheat as a template, designing gene-specific primers in the 5′ UTR and 3′ UTR, and upstream and downstream primers being as follows (as shown in SEQ ID NOs: 6-7):

upstream primer F2:
(SEQ ID NO: 6)
5′- TAGGAAGCCAAAGCGTTCGT-3′;
and
downstream primer R2:
(SEQ ID NO: 7)
5′- TACTCGTGGCGATCCATTCG-3′;

performing PCR amplification, and amplification conditions being the same as those in Embodiment 1 for amplifying cDNA. The specific amplified bands obtained were ligated to the pMT18-T vector for sequencing, and a full-length gDNA sequence of the TaPDIL4-1B gene in “Chinese Spring” wheat was obtained, as shown in SEQ ID NO: 2, with a size of 4075 bp from a start codon to a stop codon. The gDNA consists of 11 exons and 10 introns; starting from the 5′ end, lengths of the exons are 224 bp, 85 bp, 98 bp, 56 bp, 118 bp, 28 bp, 92 bp, 157 bp, 125 bp, 100 bp, and 488 bp, respectively, and lengths of the introns are 1131 bp, 143 bp, 85 bp, 422 bp, 96 bp, 115 bp, 216 bp, 86 bp, 122 bp, and 88 bp, respectively.

(SEQ ID NO: 2)
atattaacctgctcaccccggtcacccgagctcgcagcaaaacag
atcatggcgacccctcagatctaccgcaaaaccctcctccccgt
cctactcctgctcgcggccgcggcgctctacccagccgccgccga
cggcgacgaggtgctcgccctcacggagtccaccttcgagaagga
ggtcggccaggaccgtggcgcgctcgtcgagttctacgctccctg
gtccgtggctctccctgcccccctatctcctcgtgtcacgacgta
cttgtgtagtttgacctgcgcgtgagccgcttggatctggatccg
tggctgctttgcttcctcccctggtcgcagagctcggatctctga
tgggatcgtgcacgggttgccgctgggacggatcgtagcgaaatt
gtagctcggagtagagcagtctagatgctgctggaccgaactccc
gttggagtgggaaaatatcgggatctttagctaaaacaaacgatc
tgccttgttcacgctggcagacggaactgttttgatctccttttg
gcgtgcaccagaattggaattggtattagcctgttgagaattttg
ctgcaattctgatcccagtttttggatctcagatttgtttgaaaa
aaatctgggtacgagagtagaaacggtctacccgtagtagattcc
gatcaaatcttattttgtggtgatcttaatgcatctttggcttac
atgcgcattgtcatagcaagtaaagcgtccgctttaagggtcatc
tttgatgttgttagatatctggtgggtttttttttggatcatatt
caatggtagaatgcttatttgctttgtgtgtcagaatgaacttaa
gttttttactacttgggttgttccagtaagtgttgaactatattt
tctaccttataccataggcttgtcggtgaatcagttggtgcatcg
aactgaatatattatcaaagccaagagctgttgagctcaaaaccc
atctttcaataccagctcaggcttatgggtgacttgtttggtgca
tcaaaatcaacagtaataattcattgcaagtatgaaaacaataat
ctattgtaacgaggaaaagcttaaaccaagctatcaactgatgga
ttgcaaatcataccaagtgatccatgtaaattattcatagttgtg
gtaagtaatattatcaatatacgaaccaatcctagttgatcagag
gcaattgcatttaaaatcgtgtctactatttctgttagtctcata
tttattatcttgtttgtaaaaactatttcttccttagatgactgt
acttttctactctttggttcattatttgatgattgttttctgttt
tataggtgtggccactgcaagaagcttgctccggaatatgagaag
cttgctgcaagttttaaaaaggctaaatcagtcttgattgccaag
gtaaatttcgtgcttttgaaagtgttttgtacattaatgctataa
cattttttttgccaacaactgtataacatatactgttattagcct
ctaaattgcatagtatttgttatggcatatttaccaagtttctct
ctatccaggttgactgcgatgagcacaagagtgtgtgcagcaagt
atggagtttctggctaccccacaatccagtggtttcccaaaggtt
ccttggagcccaagaagtgagaatgcacacttttttttgatcaat
tcactttttgttataaaataaaatctgaaaacttaatgttgaact
cattgtttaggtatgagggtcaacgcactgctgaagcccttacag
aatatgttaactctgaagcaggtaaactccaacttgtggtgtagt
acattttccagccatgctgtcttaggtgggtttcaagtcagtcgg
gttctctatgaccatttaatggattatgtagaacatttaaggttt
caccacaggaaaactaagttggaagataagcatcacactgaagta
gaaattagacaattattagcacttaagcaatgtggtggcacttcc
tttgacccccttgtcacttgtgcatgtgctattggtaccattcat
cttcagttatacacttaacagttaatactggaaaggctgctggag
ttacttgtgtagcatcattatatttatatgtatatgttggtgtct
ctcattttcgttttagtcctgttgagtgttactagccttataact
tcatggtggtcctttaatctttgacatctgtctttcagcaaccaa
tgtgaagatagcagcagttccttcaagtgttgtggttctgaccga
ggaaacctttgactcagttgtccttgatgaaaccaaagatgtcct
tgttgagttctatgccccatggtaggttattatgatcagtttgct
atgcaccaaaatctacttcattgcaccactttaatggacataatt
tcatcaagtgccatttttgcttgacaggtgtggtcactgcaagag
tcttgctccggttagttgtagatctcagtaacctggcaatttcct
taataatatacttttctcatacaagaaatgtcatgcatcttcatt
tttcagctttctaactcaagtttgtaatttcttagatatatgaga
aggtggcctctgttttcaagcaagatgagggtgttgtgattgcta
atcttgatgctgacaaatacacaagcttggctgagaagtatgcac
tctgaattctctgttctaaacttctaatgacttttgtagactggt
ctcccttttttttaatgtaatttcgtgaagttttcctgaatgaac
ccaaatggaagacctgttaaatctaacacattaatctgacttacc
taccaattttggttatgcatgatatttaaccacataatggaagaa
gaattttaatcgtgtatattactttcaggtatggagtttctggtt
ttcccacattgaagttcttcccgaagggtaacaaagctggtgaag
agtatgagagcggtcgggagttagatgactttgttaagttcatta
atgagaaaagtggaactagccgtgattcgaagggtcagcttactt
cagaggttggtcacacatgagagcacactatggaagtaagctggg
taaaatgtaataatctgataaagcctatccgttgcaatttctgta
ggctgggcttgtggcaagtttggatgctcttgtcaaggaatttca
cagtgctgctgatgacaagcggaaggaaatcctctctaaaattga
agaggaggctgcgaagctcagtggtcctgctgtcaagtactaact
tctacctctcctccactgtgtttgcaagttatcaaattgttctca
gattttattttaatgttagtagtagcaatgccattgatgccctca
aacatttttcttctataaaccaggcacggaaagatctatgtgaat
gttgcaaagaagatattgcagaagggctctgactataccaagaag
gaaactgagaggcttcatcgcttgttggagaaggtgggcaacaag
aaagttgattcttatcacattatttttacttgatctccatattta
tgacctaatttcctgtttcataacatgacagtcgatcagtccttc
caaagccgatgaattcgccatcaagaagaacattctttcagcctt
ctcctcttaatggtgatgacccacgtgccccagccctgccattgt
tggggtgtagtcagtagtgcaacagtaccacctgccacaagagag
aagtgaaggaagacagagagaaatagagatcagagagatggcagt
gaaagtttagcggcgatagattatctgttctggttcaatgttgaa
caacatctgatatctgttttctctgccgttagcttttatggttac
agggtttcactttattagcagtaaaagggtttactcaagaacaaa
cagtaccaactggtgatgacattaataaatctgccatttgtgttt
tttcagtctgaggagagagttgtcacttgaacgttccaatttcaa
cagactgctaactgttctacttcgcaaaaattcaatatgagtcat
atactatatatatatatggtaccg.

### Embodiment 3: Acquisition of the Recombinant Plasmid pMWB110-TaPDIL4-1B

(1) Synthesizing primers comprising upstream and downstream homology a rms (homology arms are underlined) of a pMWB110 vector:

126100OEF:
(SEQ ID NO: 8)
TTGGTGTTACTTCTGCAGGTCGACTATGGCGACCCCTCAGATCTAC;
and
126100OER:
(SEQ ID NO: 9)
ATCGGGGAAATTCGAGCTCGGTACCCTTAAGAGGAGAAGGCTGAAAG

The cDNA obtained in Embodiment 1 was used as a template, and KOD FX DNA polymerase (item no.: KFX-101) was used as an amplifying enzyme for PCR amplification. The reaction system (20 μL) was: KOD FX (1 unit/μL), 0.4 μL; 2×PCR buffer, 10 μL; dNTP, 2.4 μL; 126100EF and 126100ER primers (10 μM), 0.6 μL; cDNA template, 1 μL; and deionized water, 5 μL. A PCR product comprising the TaPDIL4-1B gene of a homologous recombination arm of the pMWB110 vector was obtained. The PCR product was recovered using an Axygen gel recovery kit.
(2) Performing vector construction by homologous recombination using the PCR template comprising the homology arm obtained in the previous section and the pMWB110 vector. The vector was Peasy-Basic Seamless cloning and assembly kit from Beijing QuanShiJin. The reaction system was: 2×Basic assembly mix, 5 μL, and a molar ratio of the pMWB110 vector and the PCR template was 1:2. Reaction conditions included 50° C. for 15 minutes. At the end of the reaction, the centrifuge tube was placed on ice for a few seconds.
(3) Identification of the recombinant plasmids: screening for clones with insert fragments by colony PCR as follows:
\textcircled{1} picking a transformed white colony and drawing a short line on a plate, culturing the colony at 37° C. until the bacterial line is visible, and performing colony PCR reaction;
\textcircled{2} scrapping a small amount of the bacteria with a toothpick and transferring the small amount of the bacterium into 20 μL of PCR system containing the described primers for PCR reaction; the PCR reaction system including: 2×TaqPCRMasterMix, 10 μL; upstream primer 126100EF, 1 μL; downstream primer 126100ER, 1 μL; and ddH₂O, 8 μL, the PCR reaction program being as follows: pre-denaturation at 94° C. for 5 minutes; denaturation at 94° C. for 15 sec, annealing at 55° C. for 15 sec, extension at 72° C. for 1 minute and 15 seconds, 30 cycles/min; extension at 72° C. for 7 minutes; and storing at 20° C.;
\textcircled{3} placing the PCR product in 0.8% agarose for gel electrophoresis to detect whether it contains a fragment with a molecular weight of 1155 bp, that is, a fragment after the SEQ ID NO: 1 sequence is combined with primers 126100OEF and 1261000ER to verify that the vector is correctly constructed. After identifying the recombinant plasmid, a plant expression vector with the target gene is obtained.

The recombinant plasmid was sent for sequencing, using the sequencing primers 126100EF and 126100ER, and the correctly sequenced vector was designated as pMWB110-TaPDIL4-1B.

### Embodiment 4: Transfection of the Recombinant Plasmid into Agrobacterium C58C1 Sensory State

(1) Cultivation of Agrobacterium C58C1: picking a single colony of Agrobacterium and inoculating it into LYEB (60 mg/L rif) liquid medium of 3 mL, shaking culture at 28° C. overnight; taking 500 UL and inoculating it into LYEB (60 mg/L rif) liquid medium of 50 mL, shaking culture at 28° C. until OD600 is 0.6; transferring the bacterial solution into a centrifuge tube of 50 mL, and ice bath for 30 minutes; centrifuging at 5000 g for 5 minutes at 4° C.; discarding the supernatant, and resuspending the pellet in 10 mL of 0.15M NaCl; centrifuging at 5000 g for 5 minutes at 4° C.; discarding the supernatant and resuspending the pellet in 1 mL of 20 mM CaCl₂); dispensing in 100 μL/tube; freezing in liquid nitrogen for 5 minutes; and storing at −70° C.
(2) Transfer of the recombinant plasmid: adding 50 ng of the recombinant plasmid pMWB110-TaPDIL4-1B prepared in Embodiment 3 to 30 μL of Agrobacterium C58C1 sensory state and mixing well; consecutively, ice bath for 30 minutes, freezing in liquid nitrogen for 3-5 minutes, and water bath at 37° C. for 5 minutes; adding 1 mL of LYEB liquid medium, and slowly shaking culture at 28° C. for 2-4 h; centrifuging with 5000 rpm at 4° C.; discarding a part of the supernatant, using the remaining supernatant to resuspend the bacteria, spreading the resuspension on YEB (60 mg/Lrif) solid medium, and culturing at 28° C. for 2-3 days.
(3) Bacteriophage PCR identification: picking positive clones to obtain recombinant Agrobacterium to obtain Agrobacterium C58C1 comprising the recombinant plasmid, the specific identification steps of which are the same as step (4) in Embodiment 3.

### Embodiment 5: Screening and Obtaining Transgenic Wheat

The process is completed by the biological company.

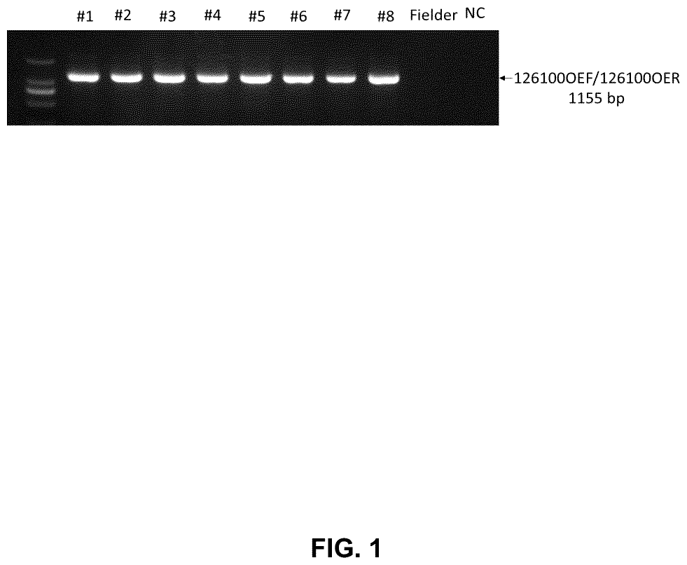

The TaPDIL4-1B gene was introduced into the wheat variety Fielder using an Agrobacterium-mediated manner to enable overexpression of the gene in wheat (this process was carried out by a commercial biotechnology company). Transgenic strains were screened for positive plants using a following PCR system including: 2×TaqPCRMasterMix, 10 μL, upstream primer 126100EF, 1 μL, downstream primer 126100ER, 1 μL, and ddH₂O, 8 μL. The PCR reaction program was as follows: pre-denaturation at 94° C. for 5 minutes; denaturation at 94° C. for 15 sec, annealing at 55° C. for 15 sec, extension at 72° C. for 1 minute and 15 seconds, 30 cycles/min; extension at 72° C. for 7 minutes; and storing at 20° C. The amplified product was subjected to gel electrophoresis, and positive plants were identified by the appearance of a specific band of 1155 bp in the gel electrophoresis (FIG. 1).

### Embodiment 6: Functional Identification of Transgenic Wheat Comprising the TaPDIL4-1B Gene for Resistance to FHB

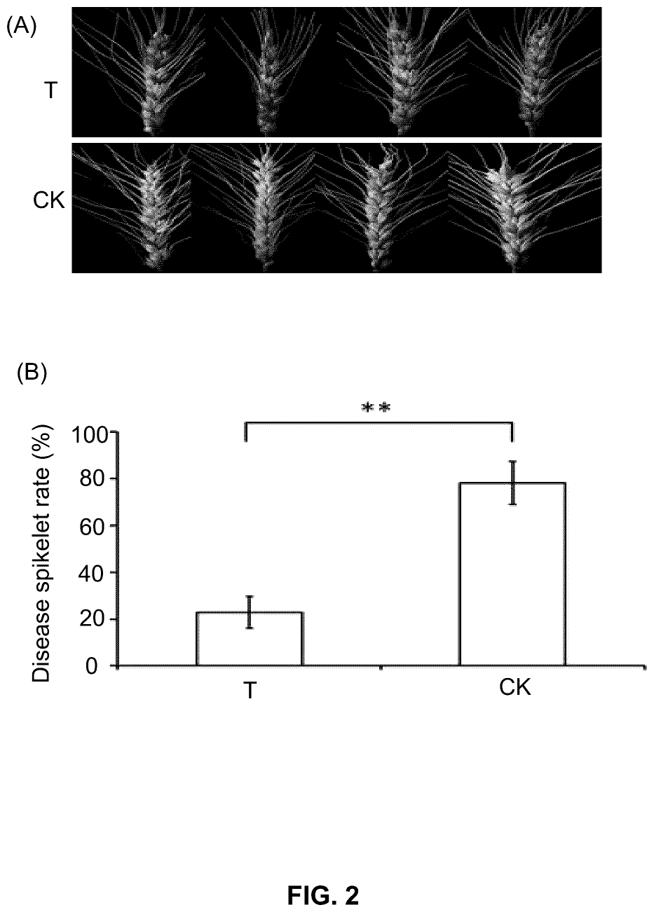

In order to verify the function of the gene, T2 generation transgenic plants overexpressing the TaPDIL4-1B gene were used for the functional identification of resistance to FHB. Phenotypic identification result showed that 21 days after single-flower drop-inoculation, the FHB resistance of the transgenic plants was significantly improved compared to that of the wild-type Fielder (FIG. 2A). The disease spikelet rate of the transgenic plants was 23.0%, while that of the wild-type Fielder was 78.1%, the difference is extremely significant (FIG. 2B), indicating that overexpression of the TaPDIL4-1B gene significantly improved FHB resistance of wheat.

In summary, the transfer of the TaPDIL4-1B gene into plants is capable of providing the plants with strong resistance to FHB.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

What is claimed is:

1. A method for constructing a transgenic plant comprising a TaPDIL4-1B gene, wherein a cDNA sequence of the TaPDIL4-1B gene is shown in SEQ ID NO: 1.

2. The method according to claim 1, wherein the plant is wheat.

3. The method according to claim 2, further comprising: introducing the TaPDIL4-1B gene or a recombinant plasmid comprising the TaPDIL4-1B gene into cells, tissues, or organs of the wheat to obtain a new wheat variety with resistance to fusarium head blight.

4. The method according to claim 3, wherein the recombinant plasmid is pMWB110-TaPDIL4-1B.

5. The method according to claim 4, further comprising: selectively labeling the recombinant plasmid pMWB110-TaPDIL4-1B.


SEQUENCE LISTING (9 sequences)

SEQ ID NO: 1 - DNA, 1104 bp, Triticum aestivum
  source 1..1104: mol_type = genomic DNA
atggcgacccctcagatctaccgcaaaaccctcctccccgtcctactcctgctcgcggccgcggcgctctacccagccgccgccgacggcgacgaggtgctcgccctcacggagtccaccttcgagaaggaggtcggccaggaccgtggcgcgctcgtcgagttctacgctccctggtgtggccactgcaagaagcttgctccggaatatgagaagcttgctgcaagttttaaaaaggctaaatcagtcttgattgccaaggttgactgcgatgagcacaagagtgtgtgcagcaagtatggagtttctggctaccccacaatccagtggtttcccaaaggttccttggagcccaagaagtatgagggtcaacgcactgctgaagcccttacagaatatgttaactctgaagcagcaaccaatgtgaagatagcagcagttccttcaagtgttgtggttctgaccgaggaaacctttgactcagttgtccttgatgaaaccaaagatgtccttgttgagttctatgccccatggtgtggtcactgcaagagtcttgctccgatatatgagaaggtggcctctgttttcaagcaagatgagggtgttgtgattgctaatcttgatgctgacaaatacacaagcttggctgagaagtatggagtttctggttttcccacattgaagttcttcccgaagggtaacaaagctggtgaagagtatgagagcggtcgggagttagatgactttgttaagttcattaatgagaaaagtggaactagccgtgattcgaagggtcagcttacttcagaggctgggcttgtggcaagtttggatgctcttgtcaaggaatttcacagtgctgctgatgacaagcggaaggaaatcctctctaaaattgaagaggaggctgcgaagctcagtggtcctgctgtcaagcacggaaagatctatgtgaatgttgcaaagaagatattgcagaagggctctgactataccaagaaggaaactgagaggcttcatcgcttgttggagaagtcgatcagtccttccaaagccgatgaattcgccatcaagaagaacattctttcagccttctcctcttaa

SEQ ID NO: 2 - DNA, 4075 bp, Triticum aestivum
  source 1..4075: mol_type = genomic DNA
atattaacctgctcaccccggtcacccgagctcgcagcaaaacagatcatggcgacccctcagatctaccgcaaaaccctcctccccgtcctactcctgctcgcggccgcggcgctctacccagccgccgccgacggcgacgaggtgctcgccctcacggagtccaccttcgagaaggaggtcggccaggaccgtggcgcgctcgtcgagttctacgctccctggtccgtggctctccctgccctccctatctcctcgtgtcacgacgtacttgtgtagtttgacctgcgcgtgagccgcttggatctggatccgtggctgctttgcttcctcccctggtcgcagagctcggatctctgatgggatcgtgcacgggttgccgctgggacggatcgtagcgaaattgtagctcggagtagagcagtctagatgctgctggaccgaactcccgttggagtgggaaaatatcgggatctttagctaaaacaaacgatctgccttgttcacgctggcagacggaactgttttgatctccttttggcgtgcaccagaattggaattggtattagcctgttgagaattttgctgcaattctgatcccagtttttggatctcagatttgtttgaaaaaaatctgggtacgagagtagaaacggtctacccgtagtagattccgatcaaatcttattttgtggtgatcttaatgcatctttggcttacatgcgcattgtcatagcaagtaaagcgtccgctttaagggtcatctttgatgttgttagatatctggtgggtttttttttggatcatattcaatggtagaatgcttatttgctttgtgtgtcagaatgaacttaagttttttactacttgggttgttccagtaagtgttgaactatattttctaccttataccataggcttgtcggtgaatcagttggtgcatcgaactgaatatattatcaaagccaagagctgttgagctcaaaacccatctttcaataccagctcaggcttatgggtgacttgtttggtgcatcaaaatcaacagtaataattcattgcaagtatgaaaacaataatctattgtaacgaggaaaagcttaaaccaagctatcaactgatggattgcaaatcataccaagtgatccatgtaaattattcatagttgtggtaagtaatattatcaatatacgaaccaatcctagttgatcagaggcaattgcatttaaaatcgtgtctactatttctgttagtctcatatttattatcttgtttgtaaaaactatttcttccttagatgactgtacttttctactctttggttcattatttgatgattgttttctgttttataggtgtggccactgcaagaagcttgctccggaatatgagaagcttgctgcaagttttaaaaaggctaaatcagtcttgattgccaaggtaaatttcgtgcttttgaaagtgttttgtacattaatgctataacattttttttgccaacaactgtataacatatactgttattagcctctaaattgcatagtatttgttatggcatatttaccaagtttctctctatccaggttgactgcgatgagcacaagagtgtgtgcagcaagtatggagtttctggctaccccacaatccagtggtttcccaaaggttccttggagcccaagaagtgagaatgcacactttgtttttgatcaattcactttttgttataaaataaaatctgaaaacttaatgttgaactcattgtttaggtatgagggtcaacgcactgctgaagcccttacagaatatgttaactctgaagcaggtaaactccaacttgtggtgtagtacattttccagccatgctgtcttaggtgggtttcaagtcagtcgggttctctatgaccatttaatggattatgtagaacatttaaggtttcaccacaggaaaactaagttggaagataagcatcacactgaagtagaaattagacaattattagcacttaagcaatgtggtggcacttcctttgacccccttgtcacttgtgcatgtgctattggtaccattcatcttcagttatacacttaacagttaatactggaaaggctgctggagttacttgtgtagcatcattatatttatatgtatatgttggtgtctctcattttcgttttagtcctgttgagtgttactagccttataacttcatggtggtcctttaatctttgacatctgtctttcagcaaccaatgtgaagatagcagcagttccttcaagtgttgtggttctgaccgaggaaacctttgactcagttgtccttgatgaaaccaaagatgtccttgttgagttctatgccccatggtaggttattatgatcagtttgctatgcaccaaaatctacttcattgcaccactttaatggacataatttcatcaagtgccatttttgcttgacaggtgtggtcactgcaagagtcttgctccggttagttgtagatctcagtaacctggcaatttccttaataatatacttttctcatacaagaaatgtcatgcatcttcatttttcagctttctaactcaagtttgtaatttcttagatatatgagaaggtggcctctgttttcaagcaagatgagggtgttgtgattgctaatcttgatgctgacaaatacacaagcttggctgagaagtatgcactctgaattctctgttctaaacttctaatgacttttgtagactggtctcccttttttttaatgtaatttcgtgaagttttcctgaatgaacccaaatggaagacctgttaaatctaacacattaatctgacttacctaccaattttggttatgcatgatatttaaccacataatggaagaagaattttaatcgtgtatattactttcaggtatggagtttctggttttcccacattgaagttcttcccgaagggtaacaaagctggtgaagagtatgagagcggtcgggagttagatgactttgttaagttcattaatgagaaaagtggaactagccgtgattcgaagggtcagcttacttcagaggttggtcacacatgagagcacactatggaagtaagctgggtaaaatgtaataatctgataaagcctatccgttgcaatttctgtaggctgggcttgtggcaagtttggatgctcttgtcaaggaatttcacagtgctgctgatgacaagcggaaggaaatcctctctaaaattgaagaggaggctgcgaagctcagtggtcctgctgtcaagtactaacttctacctctcctccactgtgtttgcaagttatcaaattgttctcagattttattttaatgttagtagtagcaatgccattgatgccctcaaacatttttcttctataaaccaggcacggaaagatctatgtgaatgttgcaaagaagatattgcagaagggctctgactataccaagaaggaaactgagaggcttcatcgcttgttggagaaggtgggcaacaagaaagttgattcttatcacattatttttacttgatctccatatttatgacctaatttcctgtttcataacatgacagtcgatcagtccttccaaagccgatgaattcgccatcaagaagaacattctttcagccttctcctcttaatggtgatgacccacgtgccccagccctgccattgttggggtgtagtcagtagtgcaacagtaccacctgccacaagagagaagtgaaggaagacagagagaaatagagatcagagagatggcagtgaaagtttagcggcgatagattatctgttctggttcaatgttgaacaacatctgatatctgttttctctgccgttagcttttatggttacagggtttcactttattagcagtaaaagggtttactcaagaacaaacagtaccaactggtgatgacattaataaatctgccatttgtgttttttcagtctgaggagagagttgtcacttgaacgttccaatttcaacagactgctaactgttctacttcgcaaaaattcaatatgagtcatatactatatatatatatggtaccg

SEQ ID NO: 3 - protein, 367 aa, Triticum aestivum
MATPQIYRKTLLPVLLLLAAAALYPAAADGDEVLALTESTFEKEVGQDRGALVEFYAPWCGHCKKLAPEYEKLAASFKKAKSVLIAKVDCDEHKSVCSKYGVSGYPTIQWFPKGSLEPKKYEGQRTAEALTEYVNSEAATNVKIAAVPSSVVVLTEETFDSVVLDETKDVLVEFYAPWCGHCKSLAPIYEKVASVFKQDEGVVIANLDADKYTSLAEKYGVSGFPTLKFFPKGNKAGEEYESGRELDDFVKFINEKSGTSRDSKGQLTSEAGLVASLDALVKEFHSAADDKRKEILSKIEEEAAKLSGPAVKHGKIYVNVAKKILQKGSDYTKKETERLHRLLEKSISPSKADEFAIKKNILSAFSS

SEQ ID NO: 4 - DNA, 20 bp, synthetic construct
  source 1..20: mol_type = other DNA
gagctcgcagcaaaacagat

SEQ ID NO: 5 - DNA, 20 bp, synthetic construct
  source 1..20: mol_type = other DNA
ccgctaaactttcactgcca

SEQ ID NO: 6 - DNA, 20 bp, synthetic construct
  source 1..20: mol_type = other DNA
taggaagccaaagcgttcgt

SEQ ID NO: 7 - DNA, 20 bp, synthetic construct
  source 1..20: mol_type = other DNA
tactcgtggcgatccattcg

SEQ ID NO: 8 - DNA, 46 bp, synthetic construct
  source 1..46: mol_type = other DNA
ttggtgttacttctgcaggtcgactatggcgacccctcagatctac

SEQ ID NO: 9 - DNA, 47 bp, synthetic construct
  source 1..47: mol_type = other DNA
atcggggaaattcgagctcggtacccttaagaggagaaggctgaaag